(19) United States
(12) Patent Application Publication
(10) Pub. No.: US 2012/0178175 A1
(43) Pub. Date: Jul. 12, 2012

(54) CWB conductivity monitor

(76) Inventor: Jay Clifford Crosman, Mamaroneck, NY (US)

(21) Appl. No.: 12/927,978

(22) Filed: Jan. 12, 2011

(51) Int. Cl. G01N 33/00

(52) U.S. Cl. 436/133; 422/69

(57) ABSTRACT

This invention is a method and apparatus for monitoring the concentration of carbon dioxide dissolved in water by means of conductivity. It distinguishes between the conductivity resulting from carbon dioxide and the conductivity resulting from other constituents dissolved in water. It can be used to monitor the quality of demineralized water, boiler feedwater, steam, or condensate in electric power generation and other industrial facilities. It is constructed by adding a column containing weak base anion exchange resin and a conductivity instrument to a typical cation conductivity monitor. A sample of the water to be monitored flows first through a typical cation conductivity monitor, then through a weak base anion exchange column, and then through an additional conductivity instrument. Conductivity measured at the outlet of the weak base anion exchange column will be essentially due to whatever concentration of carbon dioxide is dissolved in the sample because other dissolved constituents that affect conductivity have been essentially removed by either the cation exchange resin that is part of a typical cation conductivity monitor, or by the weak base anion exchange resin. By subtracting the value of conductivity due to carbon dioxide (at the outlet of the weak base anion exchange column) from the value of cation conductivity (at the outlet of the cation exchange column), the value of degassed cation conductivity is obtained. In the title of the invention, CWB conductivity is an abbreviation for cation—weak base conductivity.

In combination with existing methods for oxidizing organic compounds dissolved in water, this invention is also a method and apparatus for monitoring the concentration of dissolved or total organic carbon in water by means of conductivity. It distinguishes between the conductivity resulting from organic carbon and the conductivity resulting from inorganic constituents dissolved in water including carbon dioxide.

### TECHNICAL FIELD

Water quality monitoring in electric power generation and other industrial facilities

The electrical conductivity of absolutely pure water is extremely low—approximately 0.055 μS/cm (micro siemens per centimeter) at 25° C. This is equivalent to an electrical resistance of approximately 18.2 million ohms per centimeter at 25° C. A temperature is stated because conductivity and resistance vary with temperature.

When an inorganic salt, such as sodium chloride, is dissolved in water, the conductivity increases. The increase in conductivity is approximately proportional to the amount of salt added. Some organic compounds, such as acetic acid, and some gases, such as carbon dioxide, also increase the conductivity of water when dissolved in it. Since conductivity is an approximate indicator of the total concentration of the various substances dissolved in water, conductivity instruments are often used to monitor water quality. These are also referred to as specific conductivity instruments or monitors.

For example, municipal water supplied to an electric power generation facility may have a specific conductivity of 300 μS/cm at 25° C. This indicates that the TDS (total dissolved solids) concentration is approximately 200 ppm (parts per million). The municipal water may be treated by a membrane or ion exchange process to produce high purity demineralized water with a specific conductivity of 0.1 μS/cm or less at 25° C. to be suitable for use in the facility. A specific conductivity of 0.1 μS/cm or less at 25° C. indicates that the TDS concentration has been reduced to approximately 0.025 ppm. Both the municipal and the demineralized water qualities may be monitored by conductivity instruments.

After the demineralized water is produced, it is often stored in a tank. If the tank is open to atmosphere, carbon dioxide from the air dissolves in the demineralized water and the conductivity tends to increase from 0.1 to approximately 1.0 μS/cm at 25° C. An operator of the electric power generation facility may not know whether the higher conductivity is caused only by carbon dioxide or also by some other impurity that will have more significant consequences when the demineralized water is used in the facility. For that reason, a monitor that distinguishes between the conductivity resulting from carbon dioxide and the conductivity resulting from other constituents is useful.

An example similar to the above is the monitoring of steam turbine condensate in a Rankine cycle electric power generation facility.

In a Rankine cycle electric power generation facility, water is evaporated into steam in a boiler, steam passes through a turbine, steam is condensed in a condenser, and condensate is pumped back to the boiler. Condensation of steam in a condenser is often accomplished by relatively cold water flowing through the tube side of the condenser. Relatively cold water on the tube side absorbs heat and causes steam on the shell side to condense. Makeup water added to compensate for losses from the cycle is usually high purity demineralized water as described in previous the example above.

Water and steam in the cycle must be very pure and the chemistry must be controlled to minimize corrosion and deposition of impurities in the boiler, steam turbine, condenser, piping, and other components. Monitoring of steam and water chemistry is therefore an important aspect of the operation of a Rankine cycle electric power generation facility.

Impurities may be present in steam turbine condensate as a result of the following:
1. Cooling water could leak from the tube to the shell side of the condenser. Since cooling water generally has a much higher TDS concentration than condensate, the TDS concentration of the condensate will be increased. This increase is mostly a result of dissolved inorganic salts in the cooling water.
2. A higher than allowable concentration of impurities may be present in demineralized makeup water added to the cycle.
3. Air could leak into the shell side of the condenser and carbon dioxide could dissolve in the condensate. This can occur because the shell side of a condenser in a Rankine cycle electric power generation facility is often maintained under a partial vacuum.
4. Organic chemicals added to the cycle to control steam and water chemistry can break down because of high temperatures in the cycle. This may result in measurable concentrations of dissolved organic compounds such as acetic or formic acid. This can also occur if organic compounds are not sufficiently removed from makeup water added to the cycle.

Any of the above could affect the specific conductivity of steam turbine condensate.

The conductivity of steam turbine condensate is also affected by chemicals that are intentionally added to water in the cycle. Ammonia or other amines, such as morpholine, are often added to elevate the pH and thereby minimize the corrosion of steel surfaces in contact with water and steam in the cycle. These evaporate with water in the boiler, pass through the turbine, condense with the steam, and are present throughout the cycle. The specific conductivity resulting from chemicals intentionally added to the cycle is often approximately 2-3 μS/cm at 25° C.

To distinguish between the conductivity resulting from ammonia or other amines intentionally added and the conductivity resulting from other impurities described above, the quality of condensate is often monitored by means of a cation conductivity instrument. A sample of condensate first passes through a column containing cation exchange resin and then through a conductivity instrument.

Ammonia is present as gas (NH₃) in equilibrium with ammonium hydroxide (NH₄OH). Ammonium hydroxide dissociates into cations and anions in solution (NH₄OH [symbol] NH₄⁺¹+OH⁻¹). When a sample of condensate passes through cation exchange resin that is in the H⁺¹ form, NH₄⁺¹ will be removed from the water and replaced by H⁺¹. In the water, H⁺¹ will combine with OH⁻¹ to form additional water molecules. When some Of the NH₄⁺¹ is removed, some of the NH₃ will convert to NH₄OH. In this way, essentially all of the ammonia can be removed by cation exchange resin. A similar process occurs when other amines are present.

If cooling water is leaking from the tube to the shell side of a condenser, and sodium chloride is present in the condensate sample being monitored, it dissociates into cations and anions also (NaCl [symbol] Na⁺¹+Cl⁻¹). When the sample of condensate passes through cation exchange resin, sodium will be removed and replaced with H⁺¹ in solution. Cl⁻¹ will combine with H⁺¹ to form dilute hydrochloric acid (HCl). Since the conductivity of dilute hydrochloric acid is slightly higher than the conductivity of the same concentration of dilute sodium chloride, the dilute hydrochloric acid will produce a reading in a conductivity monitor that is similar to the reading that would have occurred if the sodium had not been removed. The same applies to other inorganic salts that may be present as a result of cooling water leakage into the shell side of a condenser.

Carbon dioxide and organic acids will not be removed by the cation exchange resin.

If the specific conductivity of condensate is 3 μS/cm at 25° C., a facility operator may not know what portion of this is caused by ammonia or other amines, and what portion may be caused by impurities. If a cation conductivity instrument is used, ammonia or other amines are removed, and the cation conductivity value will be noticeably different if impurities are present. A typical requirement for condensate cation conductivity in an electric power generation facility is 0.1 μS/cm at 25° C.

Many Rankine cycle electric power generation plants include a deaerator to remove dissolved gases, including carbon dioxide, from the condensate or feedwater before it goes to the boiler. For that reason, the presence of carbon dioxide is not as significant as the presence of other impurities in the condensate. For example, the impurities that result from cooling water leakage into the condensate (sodium chloride or other inorganic salts of sodium, potassium, calcium, and magnesium) could damage the boiler and turbine. Unlike carbon dioxide, these impurities are not removed by a deaerator. It would be useful to the operator of an electric power generation facility to know whether a high cation conductivity value indicates the presence of carbon dioxide or another impurity.

The considerations regarding condensate described above apply equally as well to boiler feedwater and steam in electric power generation and many other industrial facilities.

For reasons described above, some Rankine cycle electric power generation plants include degassed cation conductivity monitors. The purpose of a degassed cation conductivity monitor is to remove carbon dioxide, but not inorganic anions such as chlorides, sulfates, or nitrates. Degassed cation conductivity is measured after a cation exchange column and after the carbon dioxide is removed.

Two types of degassed cation conductivity monitors have been previously patented—a boiling type and a gas scrubber type.

A boiling type degassed cation conductivity monitor is described in U.S. Pat. No. 2,832,673 “Apparatus and Method for Determining Steam Purity” by Larson and Lane (Apr. 29, 1958). It is described in U.S. Pat. No. 4,251,219 “Apparatus for and method of determining contaminants on low pressure condensate” by Larson, et al (Feb. 17, 1981). A similar boiling type degassed cation conductivity monitor is described in U.S. Pat. No. 4,251,220 by Larson, et al. dated Feb. 17, 1981, and entitled “Apparatus for and method of determining high pressure, high temperature feedwater contaminants”.

A gas scrubber type degassed cation conductivity monitor is described in U.S. Pat. No. 3,705,477 “Condensate Analyzer” by Longo and Duff (Dec. 12, 1972).

Two commercially available degassed cation conductivity monitors of these types are described below.

### Boiling Type Degassed Cation Conductivity Monitor

Sentry Equipment Corp. has offered a device entitled “DCCP—Degassed Cation Conductivity Panel”. A sample passes through a conductivity cell which measures conductivity. After passing through a bed of cation exchange resin, a second conductivity cell measures cation conductivity. The sample then passes through an electric heater which increases the temperature to near the boiling point and drives off volatile impurities such as carbon dioxide. It passes through a cooler and then through a third conductivity cell which measures degassed cation conductivity. The sample is cooled after the electric heater because the third or degassed cation conductivity measurement will be less accurate if the temperature remains near the boiling point.

A disadvantage of this type of degassed cation conductivity monitor is the electricity consumed in heating the sample to near the boiling point.

A second disadvantage is that heating generally does not remove all of the carbon dioxide. Technical papers on this subject have indicated that approximately 70% removal of carbon dioxide is typical. If 30% of the carbon dioxide remains after heating and cooling, it will affect the conductivity indicated by the third or degassed cation conductivity monitor, and the value of degassed cation conductivity will be inaccurate.

A third disadvantage is the need to either monitor conductivity at a high temperature or cool the sample. If the conductivity of water is measured at or near the boiling point, temperature compensation must be used to determine what the conductivity would be at 25° C. Temperature compensation introduces an additional potential inaccuracy.

A fourth disadvantage is the need to maintain an electric heating element and the components that control it.

### Gas Scrubber Type Degassed Cation Conductivity Monitor

Waters Equipment has offered a device entitled “Degassing Sparger with Nitrogen Generator”. A sample passes through a bed of cation exchange resin and then through a conductivity cell that measures cation conductivity. It then passes through a chamber with a sparger where high purity nitrogen is bubbled through the sample. The sample then flows through a conductivity cell that measures degassed cation conductivity.

Bubbling nitrogen through the sample removes carbon dioxide because the sample is in contact with a gas atmosphere that contains almost no carbon dioxide. Because carbon dioxide in the liquid sample tends to reach equilibrium with the gas atmosphere, carbon dioxide is removed from the liquid sample.

In the Waters Equipment device, nitrogen is supplied from a generator that produces it from instrument air. The process by which nitrogen is generated is not specified in literature published by Waters Equipment. It may be by membrane separation or pressure swing adsorption.

Some degassed cation conductivity monitors have used high purity nitrogen from a pressurized cylinder as shown in FIG. 2. These are generally not used continuously because of the expense and inconvenience of periodically replacing the nitrogen cylinder. The Waters Equipment unit described above is an attempt to overcome this disadvantage by continuously generating nitrogen. It, however, has the disadvantage that the nitrogen generator increases the cost, complexity, and maintenance requirements of the instrument.

### Background Art in Relation to Dissolved or Total Organic Carbon Monitoring

A number of different devices for monitoring the concentration of TOC (total organic carbon) in a sample of water are commercially available. Essentially all of these include the following:
Acidification of the sample
Oxidation of organic compounds in the sample
Measurement of the amount of carbon dioxide produced by oxidation of organic compounds

Acidification of the sample can be accomplished by adding an acid or by passing the sample through a column with cation exchange resin. Passing a sample through a column with cation exchange resin acidifies the water sample because cations such as calcium, magnesium, and sodium are removed. Anions such as chlorides and sulfates remain in solution, and these anions form acids.

In some TOC monitors, the sample is acidified to allow carbon dioxide that is in solution to be removed by contacting the sample with carbon dioxide free gas. By removing carbon dioxide from the sample, the carbon dioxide concentration in solution after oxidation will only be a result of organic compounds that have been converted to carbon dioxide.

Published literature for one TOC monitor indicates that oxidation of organic compounds is accomplished by exposure of the sample to UV light. Published literature for another TOC monitor indicates that oxidation of organic compounds is accomplished by addition of sodium persulfate, a strong oxidizing agent, and exposure to UV light.

When organic compounds are oxidized, the carbon that was part of these compounds is converted to carbon dioxide. Since the atomic weight of carbon is 12 and the molecular weight of carbon dioxide is 44, the total amount of carbon is 27.3% of the total amount of carbon dioxide produced by oxidation.

According to some published literature for TOC monitors, the amount of carbon dioxide produced by oxidation of the organic compounds is measured by differential conductivity—the difference between the conductivity before oxidation and the conductivity after oxidation. This method assumes that the organic compounds do not result in any conductivity and that the conductivity after oxidation will indicate how much additional carbon dioxide is now dissolved in the water.

Although some organic compounds do not result in any conductivity, others such as acetic acid do. When some organic compounds are oxidized and removed from solution, there will be no decrease in conductivity. When other organic compounds are oxidized and removed from solution, there will be a decrease in conductivity. For that reason, the increase in conductivity after oxidation may not only be a result of additional carbon dioxide in solution. It may sometimes be decreased by organic compounds that are removed by the oxidation. For that reason, differential conductivity before and after oxidation may not be an accurate indication of the concentration of the concentration of organic carbon dissolved in a sample of water.

According to published literature for other TOC monitors, the amount of carbon dioxide produced by oxidation of the organic compounds is measured by carrying the sample in a carbon dioxide free gas stream, and then passing it through an NDIR (non-dispersive infra red) detector. In the NDIR detector, carbon dioxide is measured directly and the potential inaccuracy of the differential conductivity is avoided.

Disadvantages of this type of device are the initial cost of the NDIR detector and the operating cost of supplying carbon dioxide free carrier gas.

### SUMMARY OF INVENTION

The technical problem is described above under “Background Art in Relation to Monitoring the Concentration of Carbon Dioxide and Degassed Cation Conductivity” and “Background Art in Relation to Dissolved or Total Organic Carbon Monitoring”. Each of the existing devices used to monitor these parameters has one or more disadvantages which include high initial cost, high operating cost, low accuracy, low reliability, or high maintenance requirements.

Objects of this invention are to provide improved methods and apparatus for monitoring the following: the concentration of carbon dioxide dissolved in a sample of water, the degassed cation conductivity of water, and the concentration of dissolved or total organic compounds in water. These improved methods and apparatus provide higher reliability, higher accuracy, lower initial cost, lower operating cost, and lower maintenance.

This invention consists of the addition of a column of weak base anion exchange resin and a conductivity instrument to a typical cation conductivity instrument. In a CWB conductivity monitor, a sample of the water to be monitored passes through a column with cation exchange resin, a conductivity instrument, a column with weak base anion exchange resin, and another conductivity instrument. If the sample is high temperature water, it is first cooled. If the sample is steam, it is first condensed and cooled. This invention can be used with a specific conductivity instrument upstream of the column with cation exchange resin.

If the sample to be monitored contains ammonia or other amines, these will be removed when the sample passes through the column with cation exchange resin. If inorganic salts such as sodium chloride are present, the cationic part of these will be removed when the sample passes through the column with cation exchange resin. A conductivity instrument downstream from the column of cation exchange resin will indicate cation conductivity.

When the sample passes through the column with weak base anion exchange resin, anions such as chloride, sulfate, and nitrate will be removed. Organic compounds such as acetic and formic acid will be mostly removed. Some organic compounds will not be removed. If silica is present, it will not be removed.

At the outlet of the weak base anion exchange column the remaining constituents will be essentially carbon dioxide, silica, and any organic compounds that were not removed by the weak base anion exchange resin. Since silica and the remaining organic compounds will not have a significant effect on the conductivity, the value of conductivity after the column of weak base anion exchange resin will be almost entirely due to carbon dioxide.

The concentration of carbon dioxide in the water being sampled can then be determined by means of a graph such as the one shown in FIG. 6. The unit for CO₂ concentration (ppb) refers to parts per billion. The unit for conductivity (μmho) is apparently an abbreviation for μmho/cm and is the same as μS/cm.

The value of degassed cation conductivity can monitored by subtracting the conductivity of the sample downstream of the weak base anion exchange column (conductivity resulting only from carbon dioxide) from the value of the conductivity downstream of the cation exchange column (cation conductivity). This can be accomplished by subtracting the values manually, or by subtracting the values by means of an electronic device. If the values are subtracted by an electronic device, the value of degassed cation conductivity can be continuously displayed. The concentration of carbon dioxide can likewise be calculated electronically and continuously displayed.

### DESCRIPTION OF DRAWINGS

FIG. 1 illustrates the design of a typical boiling type degassed cation conductivity monitor.

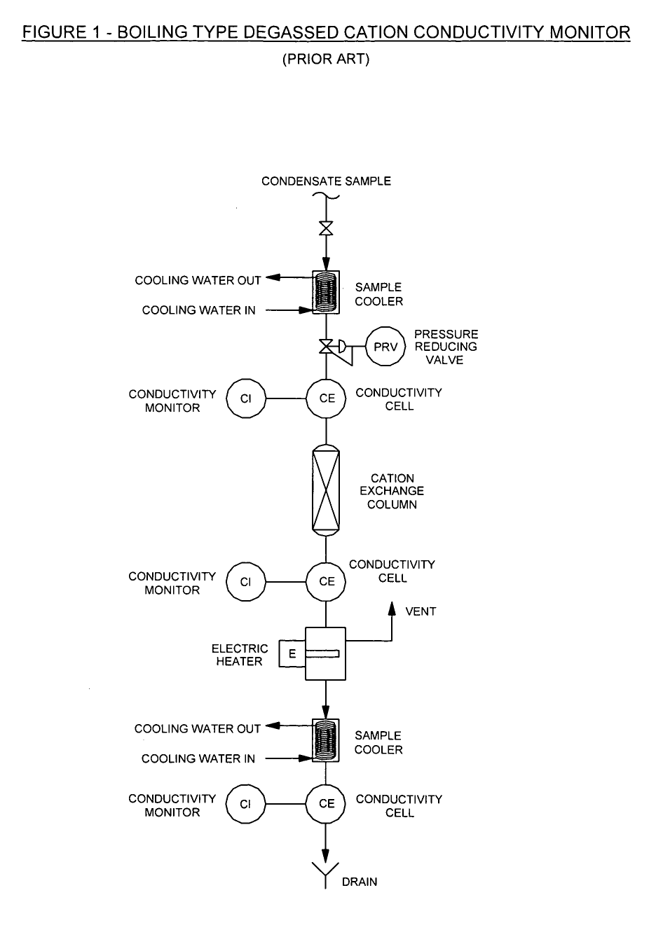

FIG. 2 illustrates the design of a typical gas scrubber type degassed cation conductivity monitor.

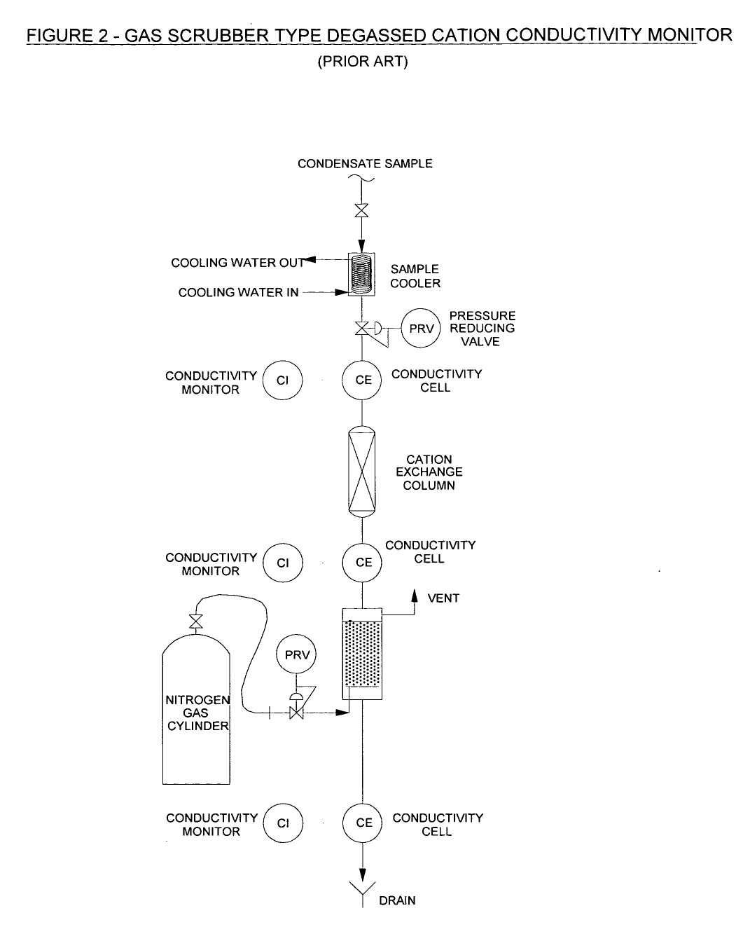

FIG. 3 illustrates a CWB conductivity monitor that can be used to monitor the concentration of carbon dioxide dissolved in a sample of water or degassed cation conductivity.

FIG. 4 illustrates a CWB conductivity monitor in combination with an existing method for oxidizing organic compounds dissolved in water. It illustrates a CWB conductivity monitor for monitoring the concentration of dissolved organic carbon dissolved in water by means of conductivity.

FIG. 5 illustrates a CWB conductivity monitor in combination with an existing method for oxidizing organic compounds dissolved in water. It illustrates a CWB conductivity monitor for monitoring the concentration of total organic carbon dissolved in water by means of conductivity.

FIG. 6 is a graph showing how CO₂ can be determined based on the conductivity of water.

### DESCRIPTION OF EMBODIMENTS

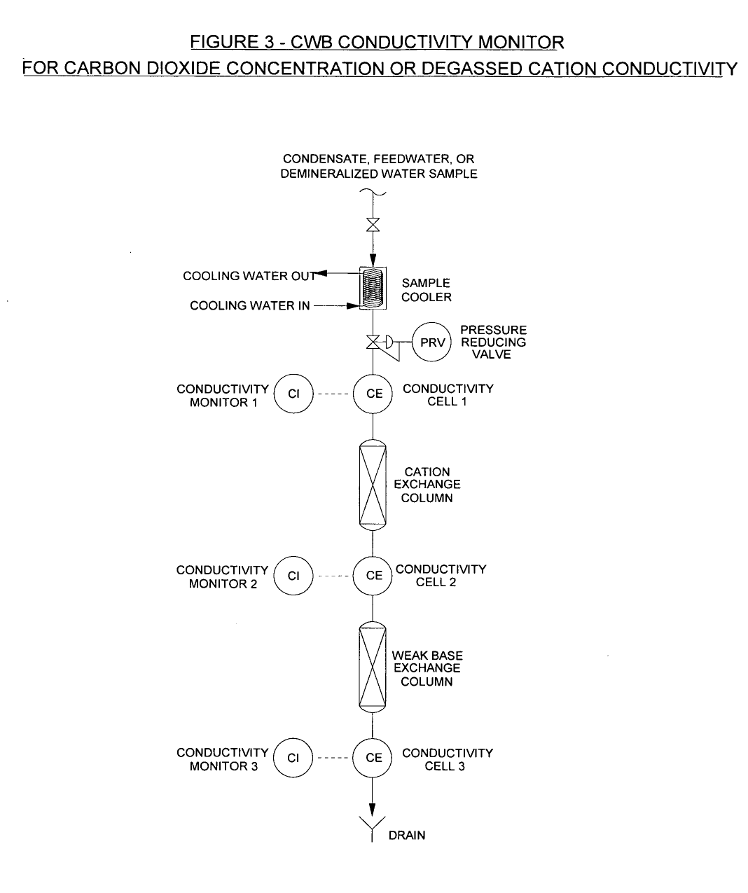

A preferred embodiment of this invention for monitoring the concentration of carbon dioxide and degassed cation conductivity is illustrated in FIG. 3.

The sample cooler, pressure reducing valve, conductivity cell 1 and conductivity monitor 1, cation exchange column, conductivity cell 2, and conductivity monitor 2 illustrate specific and cation conductivity apparatus in common use.

As shown on FIG. 3, the sample continues through a column of weak base anion exchange resin after passing through conductivity cell 2. The size of the column and the volume of weak base anion exchange resin in it can be approximately the same as the cation exchange column. The column can be constructed in the same way as cation exchange columns that are commonly used for cation conductivity apparatus.

As shown on FIG. 3, the sample then continues through conductivity cell 3.

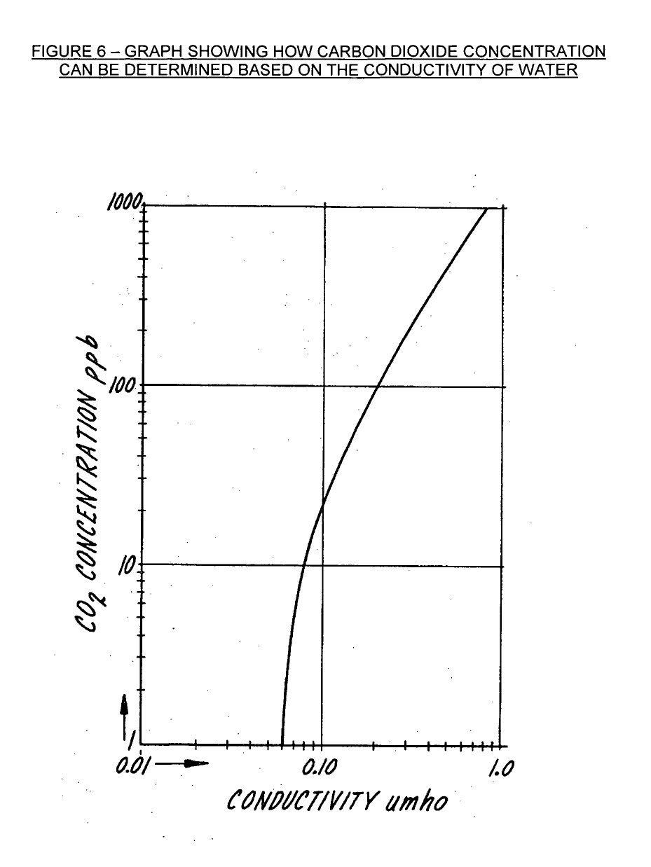

Since the impurities that have a significant effect on conductivity (other than carbon dioxide) have essentially been removed, the value indicated by conductivity monitor 3 will be essentially due to carbon dioxide. The concentration of carbon dioxide in the sample can be obtained from the value of conductivity monitor 3. For example, if the conductivity downstream of the weak base anion exchange column is 0.2 μS/cm, the carbon dioxide concentration is approximately 100 micrograms per liter. This can be seen on the graph of carbon dioxide concentration versus conductivity on FIG. 6.

The value of degassed cation conductivity can be obtained by subtracting the value of conductivity monitor 3 from conductivity monitor 2. For example, if the value of conductivity monitor 2 is 1.2 μS/cm and the value of conductivity monitor 3 is 1.0 μS/cm, the value of CWB conductivity will be 1.2 minus 1.0 or 0.2 μS/cm. This is the same as degassed cation conductivity because it is that portion of the conductivity that is caused by anionic constituents (other than carbon dioxide) such as chlorides, sulfates, nitrates, and organic acids. It is what the cation conductivity would be if the carbon dioxide was actually removed.

The value of degassed cation conductivity can also be obtained by transmitting the values of conductivity monitors 2 and 3 to an instrument that electronically subtracts the value of conductivity monitor 3 from the value of conductivity monitor 2 and continuously displays the value of CWB or degassed cation conductivity.

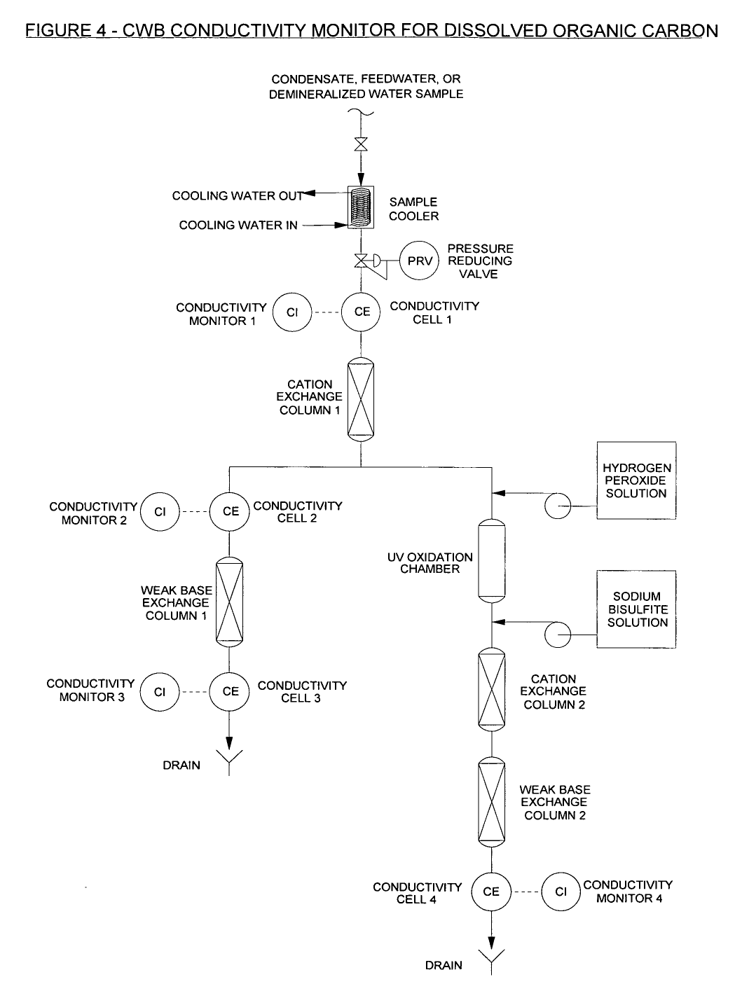

A second preferred embodiment of this invention for monitoring the concentration of organic compounds dissolved in water by means of conductivity is illustrated in FIG. 4.

As shown in FIG. 4, a sample of the water to be monitored flows through cation exchange column 1. A portion of the sample flows through conductivity cell 2 and the cation conductivity is monitored by conductivity monitor 2. This portion of the sample then flows through a column of weak base anion exchange resin and the concentration of dissolved carbon dioxide is monitored based on the value of conductivity monitor 3 as described above.

Another portion of the sample of water to be monitored is treated by addition of a solution of hydrogen peroxide (H₂O₂) and flows through a chamber where it is exposed to ultraviolet light. This will oxidize organic compounds dissolved in the water and convert these to carbon dioxide. An example is the oxidation of acetic acid as follows:

CH₃COOH+4H₂O₂→2CO₂+6H₂O

The sample is then treated by addition of a solution of sodium bisulfite, a reducing agent, to remove any excess hydrogen peroxide remaining in the sample. Reduction of excess hydrogen peroxide is needed to prevent oxidation of either the cation or weak base exchange resins downstream. Reduction by sodium bisulfite will occur according to the following:

H₂O₂+NaHSO₃→H₂O+NaHSO₄

Other oxidizing and reducing chemicals could also be used.

If excess sodium bisulfite is added, the result will be that all of the hydrogen peroxide will be converted to water. Remaining in solution will be some amount of sodium busulfite (NaHSO₃) and some amount of sodium bisulfate (NaHSO₄).

As shown on FIG. 4, the same portion of the sample then flows through cation exchange column 2. Cation exchange resin in the column will remove the sodium in solution as a result of the addition of sodium bisulfite and from the conversion of it to sodium bisulfate by reaction with hydrogen peroxide.

The same portion of the sample then flows through weak base anion column 2. Weak base anion exchange resin in this column will remove anions such as chloride, sulfate, or nitrate that were originally in the sample. It will remove sulfite remaining in solution as a result of the addition of sodium bisulfite. It will also remove sulfate remaining in solution as a result of the conversion of sulfite to sulfate by reaction with hydrogen peroxide.

After the treatment described above, all of the ammonia or other amines (if present) will have been removed from the sample by cation exchange column 1. All of the cations resulting from any inorganic impurities or from the addition of sodium busulfite will have been removed from the sample by either cation exchange column 1 or cation exchange column 2. Inorganic anions such as chloride, sulfate, sulfite, and nitrate will have been removed from the sample by weak base column 2. Essentially all of the organic impurities will have been converted to carbon dioxide and water.

The only constituents remaining in solution will be carbon dioxide and silica. Since silica will not result in a significant conductivity, the only constituent that will affect conductivity will be carbon dioxide. When the sample then flows through conductivity cell 4, the value indicted by conductivity monitor 4 will be a result of the carbon dioxide in solution.

The carbon dioxide in solution when the sample flows through conductivity cell 4 will be a combination of the carbon dioxide originally present in the sample and the carbon dioxide produced by oxidation of the organics. Since conductivity monitor 3 indicates the conductivity resulting from carbon dioxide originally present in the sample, the concentration of carbon dioxide produced by the oxidation of organics can be determined by subtracting the value of conductivity monitor 3 from the value of conductivity monitor 4.

As described above under “Background of the Invention in Relation to Organic Carbon Monitoring”, when organic compounds are oxidized, the carbon that was part of these compounds is converted to carbon dioxide. Since the atomic weight of carbon is 12 and the molecular weight of carbon dioxide is 44, the total amount of carbon is 27.3% of the total amount of carbon dioxide produced by oxidation.

Based on the above, the concentration of dissolved organic carbon in the sample can be determined by multiplying the concentration of carbon dioxide by 0.273. This could be determined manually by subtracting the value of conductivity monitor 3 from the value of conductivity monitor 4, reading a graph to determine the associated carbon dioxide concentration, and then multiplying by 0.273. This could also be accomplished by means of an electronic device that then displays dissolved organic carbon concentration continuously.

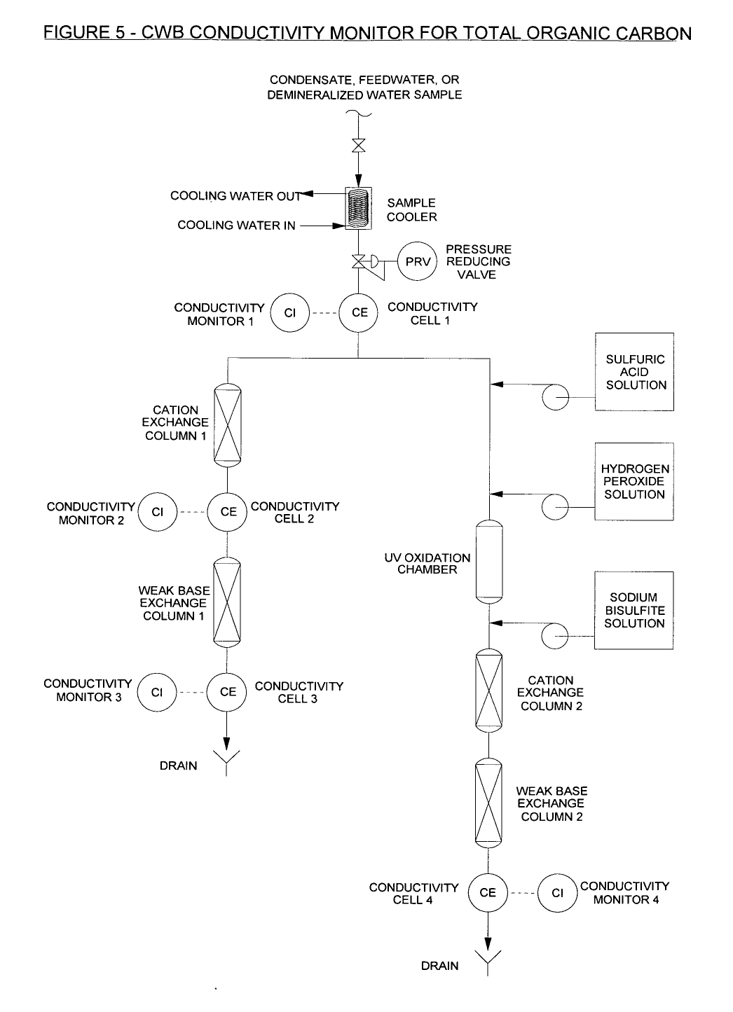

A third preferred embodiment of this invention for monitoring the concentration of organic compounds dissolved in water by means of conductivity is illustrated in FIG. 5. The second embodiment illustrated by FIG. 4 monitors dissolved organic carbon because organic constituents that are suspended in the water will be essentially removed by cation exchange column 1. FIG. 5 differs from FIG. 4 because the portion of the sample to be oxidized does not pass through a cation exchange column prior to oxidation.

Acidification of this portion of the sample is accomplished by addition of sulfuric acid. For that reason, organic constituents that are suspended as well as organic constituents that are dissolved are converted to carbon dioxide by the oxidation process.

When the sample passes through conductivity cell 4 on FIG. 5, carbon dioxide in solution will be a result of carbon dioxide originally present in the sample, carbon dioxide resulting from the oxidation of dissolved organic constituents, and carbon dioxide resulting from the oxidation of suspended organic constituents. When the carbon dioxide originally present in the sample is subtracted, the result will be carbon dioxide resulting from dissolved and suspended organic constituents, or total organic carbon. The concentration of total organic carbon can be determined from the conductivity difference in the same way as described above for dissolved organic carbon.

The following are claimed:

1. A method and apparatus to monitor the concentration of dissolved carbon dioxide in demineralized water, boiler feedwater, steam, or steam turbine condensate comprised of the following:
a cation conductivity instrument that has been in common use comprised of a column of cation exchange resin followed by a conductivity cell and monitor
a column of weak base anion exchange resin similar in size to the cation exchange resin column described above, located downstream from the above column of cation exchange resin and conductivity cell
a conductivity cell and monitor located downstream from the above column of weak base anion exchange resin
conversion of the conductivity downstream of the column of weak base anion exchange resin to a carbon dioxide concentration either manually by means of a graph showing carbon dioxide concentration versus conductivity or by an electronic device

2. A method and apparatus to monitor degassed cation conductivity in demineralized water, boiler feedwater, steam, or steam turbine condensate comprised of the following:
a cation conductivity instrument that has been in common use comprised of a column of cation exchange resin followed by a conductivity cell and monitor
a column of weak base anion exchange resin similar in size to the cation exchange resin column described above, located downstream from the above column of cation exchange resin and conductivity cell
a conductivity cell and monitor located downstream from the above column of weak base anion exchange resin
subtraction the conductivity downstream from the weak base anion exchange column from the conductivity downstream from the weak base anion exchange column either manually or by an electronic device

3. A method and apparatus to monitor the concentration of dissolved organic carbon in a sample of water comprised of the following:
passing a sample of the water to be monitored through a cation conductivity instrument that has been in common use comprised of a column of cation exchange resin followed by a conductivity cell and monitor
passing a first portion of a sample of the water to monitored through a column of weak base anion exchange resin and then through another conductivity cell and monitor
treating a second portion of a sample of the water to be monitored by addition of an oxidizing chemical, oxidizing it by ultraviolet light, or a combination of these; treating this portion of the sample by addition of a reducing agent such as sodium bisulfite; passing this portion of the sample through a column with cation exchange resin; passing this portion of the sample through a column with weak base anion exchange resin; and passing this portion of the sample through another conductivity cell and monitor
subtraction of the conductivity downstream from the column of weak base anion exchange resin in the first portion described above from the conductivity downstream of the column of weak base anion exchange resin in the second portion of the sample described above to obtain the conductivity of carbon dioxide resulting from oxidation of organic constituents, and then converting this, either manually or by an electronic device, to a concentration of carbon
